(19) United States
(12) Patent Application Publication
(10) Pub. No.: US 2022/0223290 A1
(43) Pub. Date: Jul. 14, 2022

(54) MEANS AND METHODS FOR ASSESSING SPINAL MUSCULAR ATROPHY (SMA)

(71) Applicant: Hoffmann-La Roche Inc., Little Falls, NJ (US)

(72) Inventors: Michael LINDEMANN, Schopfheim (DE); Florian LIPSMEIER, Basel (CH); Cedric Andre Marie Vincent Geoffrey SIMILLION, Lutzelfluh-Goldbach (CH)

(73) Assignee: Hoffmann-La Roche Inc., Little Falls, NJ (US)

(21) Appl. No.: 17/708,385

(22) Filed: Mar. 30, 2022

Related U.S. Application Data
(63) Continuation of application No. PCT/EP2020/077211, filed on Sep. 29, 2020.

(30) Foreign Application Priority Data
Sep. 30, 2019 (EP) 19200577.5

(51) Int. Cl. G16H 50/20; G01N 33/68

(52) U.S. Cl. CPC G16H 50/20; G01N 2800/285; G01N 33/6896

(57) ABSTRACT

The present invention relates to the field of disease tracking. Specifically, it relates to a method for predicting the forced vital capacity (FVC) in a subject suffering from spinal muscular atrophy (SMA) comprising the steps of determining at least one performance parameter from a dataset of measurements of central motor function capabilities from said subject, comparing the determined at least one performance parameter to a reference obtained from a computer-implemented regression model generated on training data using partial least-squares (PLS) analysis with the at least one performance parameters, and predicting the FVC of the subject based on said comparison. The present invention also relates to a mobile device and/or a remote device as well as software which is tangibly embedded to one of the devices and which carries out the method of the invention, wherein said mobile device and said remote device are operatively linked to each other.

### CROSS REFERENCE TO RELATED APPLICATIONS

This application is a continuation of International Application No. PCT/EP2020/077211, filed Sep. 29, 2020, which claims priority to EP Application No. 19200577.5, filed Sep. 30, 2019, which are incorporated herein by reference in their entireties

### BACKGROUND

Spinal muscular atrophy (SMA) is an autosomal recessive disease also called proximal spinal muscular atrophy and 5 q spinal muscular atrophy. It is a life-threatening, neuromuscular disorder with low prevalence associated with loss of motor neurons and progressive muscle wasting.

The disorder is caused by a genetic defect in the SMN1 gene (Brzustowicz, 1990, Lefebvre 1995). This gene encodes the SMN protein which is wide-spread expressed in all eukaryotic cells and necessary for survival of motor neurons. Reduced levels of the protein result in loss of function of neuronal cells in the anterior horn of the spinal cord. As a consequence of the loss of neuronal function, atrophy of skeletal muscles occurs.

Spinal muscular atrophy manifests in various degrees of severity, which all have in common progressive muscle wasting and mobility impairment. Proximal muscles and respiratory muscles are affected first. Other body systems may be affected as well, particularly in early-onset forms of the disorder. SMA is the most common genetic cause of infant death.

Four different types of SMA are described. Four different types of SMA are known. The infantile SMA or SMA1 (Werdnig-Hoffmann disease) is a severe form that manifests in the first months of life, usually with a quick and unexpected onset (“floppy baby syndrome”). The intermediate SMA or SMA2 (Dubowitz disease) affects children who are never able to stand and walk but who are able to maintain a sitting position at least some time in their life. The juvenile SMA or SMA3 (Kugelberg-Welander disease) manifests, typically, after 12 months of age and describes people with SMA3 who are able to walk without support at some time, although many later lose this ability. The adult SMA or SMA4 manifests, usually, after the third decade of life with gradual weakening of muscles that affects proximal muscles of the extremities frequently requiring the person to use a wheelchair for mobility.

For all SMA types, typical symptoms are hypotonia associated with absent reflexes, fibrillation in the electromyogram as well as muscle denervation and (sometimes) serum creatine kinase increase (Rutkove 2010).

While the above symptoms suggest SMA, the diagnosis can only be confirmed with certainty through genetic testing for bi-allelic deletion of exon 7 of the SMN1 gene. Genetic testing is usually carried out using a blood sample, and MLPA is one of more frequently used gene sequencing techniques, as it also allows establishing the number of SMN2 gene copies.

Preimplantation or prenatal genetic testing is also available for SMA. In particular, preimplantation genetic diagnosis can be used to screen for SMA-affected embryos during in-vitro fertilization. Prenatal testing for SMA is possible through chorionic villus sampling, cell-free fetal DNA analysis and other methods. However, theses genetic testing methods are only suitable if there is already suspicion for the potential development of SMA, e.g., due to the parents medical history.

So far, Nusinersen (Spinraza™) is the only approved drug for the treatment of SMA. It is a modified antisense oligonucleotide which targets the intronic splicer N1. In addition to drug treatment, patients suffering from SMA typically require special medical care, in particular with respect to orthopedics, mobility support, respiratory care, nutrition, cardiology and mental health.

The respiratory system is the most common system affected in SMA and the complications are the leading cause of death. Accordingly, the characterization of the function of the respiratory system as well as respiratory care are critical factors of clinically handling the disease. The determination of the forced vital capacity is typically carried out in order to characterize the function of the respiratory system. SMA patients with poor FVC may need respiratory support.

The forced vital capacity (FVC) is the volume of air that can forcibly be blown out after full inspiration. It is typically determined by a hospital of medical doctor's residency using spirometry devices.

However, diagnostic tools are needed that allow a reliable diagnosis and identification of the FVC in SMA patients in order to allow for proper respirator care and/or an accurate treatment.

### SUMMARY

The technical problem underlying the present invention may be seen in the provision of means and methods complying with the aforementioned needs. The technical problem is solved by the embodiments characterized in the claims and described herein below.

The present invention relates to the field of disease tracking and potentially even diagnostics. Specifically, it relates to a method for predicting the forced vital capacity (FVC) in a subject suffering from spinal muscular atrophy (SMA) comprising the steps of determining at least one performance parameter from a dataset of measurements of central motor function capabilities from said subject, comparing the determined at least one performance parameter to a reference obtained from a computer-implemented regression model generated on training data, in an embodiment using partial least-squares (PLS) analysis, with the at least one performance parameters, and predicting the FVC of the subject based on said comparison. The present invention also relates to a mobile device comprising a processor, at least one sensor and a database as well as software which is tangibly embedded to said device and, when running on said device, carries out the method of the invention as well as a system comprising a mobile device comprising at least one sensor and a remote device comprising a processor and a database as well as software which is tangibly embedded to said device and, when running on said device, carries out the method of the invention, wherein said mobile device and said remote device are operatively linked to each other. Furthermore, the invention contemplates the use of the aforementioned mobile device or system for predicting the forced vital capacity (FVC) in a subject suffering from spinal muscular atrophy (SMA) using at least one performance parameter from a dataset of measurements of central motor function capabilities from said subject.

Thus, the invention relates to a method for predicting the forced vital capacity (FVC) in a subject suffering from spinal muscular atrophy (SMA) comprising the steps of:
a) determining at least one performance parameter from a dataset of measurements of central motor function capabilities from said subject;
b) comparing the determined at least one performance parameter to a reference obtained from a computer-implemented regression model generated on training data, in an embodiment using partial least-squares (PLS) analysis, with the at least one performance parameters; and
c) predicting the FVC of the subject based on said comparison.

The method is, typically, a computer implemented method, i.e. the steps a) to c) are carried out in an automated manner by use of a data processing device. Details are also found herein below and in the accompanying Examples.

In some embodiments, the method may also comprise prior to step (a) the step of obtaining from the subject using a mobile device a dataset of measurements of central motor function capabilities from said subject during predetermined activity performed by the subject or during a predetermined time window. However, typically the method is an ex vivo method carried out on an existing dataset of measurements from a subject which does not require any physical interaction with the said subject.

The method as referred to in accordance with the present invention includes a method which essentially consists of the aforementioned steps or a method which may include additional steps.

### DETAILED DESCRIPTION

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically”, “typically”, and “more typically” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

The method may be carried out on the mobile device by the subject once the dataset of pressure measurements has been acquired. Thus, the mobile device and the device acquiring the dataset may be physically identical, i.e. the same device. Such a mobile device shall have a data acquisition unit which typically comprises means for data acquisition, i.e. means which detect or measure either quantitatively or qualitatively physical and/or chemical parameters and transform them into electronic signals transmitted to the evaluation unit in the mobile device used for carrying out the method according to the invention. The data acquisition unit comprises means for data acquisition, i.e. means which detect or measure either quantitatively or qualitatively physical and/or chemical parameters and transform them into electronic signals transmitted to the device being remote from the mobile device and used for carrying out the method according to the invention. Typically, said means for data acquisition comprise at least one sensor. It will be understood that more than one sensor can be used in the mobile device, i.e. at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors and the like. The evaluation unit typically comprises a processor and a database as well as software which is tangibly embedded to said device and, when running on said device, carries out the method of the invention. More typically, such a mobile device may also comprise a user interface, such as a screen, which allows for providing the result of the analysis carried out by the evaluation unit to a user.

Alternatively, it may be carried out on a device being remote with respect to the mobile device that has been used to acquire the said dataset. In this case, the mobile device shall merely comprise means for data acquisition, i.e. means which detect or measure either quantitatively or qualitatively physical and/or chemical parameters and transform them into electronic signals transmitted to the device being remote from the mobile device and used for carrying out the method according to the invention. Typically, said means for data acquisition comprise at least one sensor. It will be understood that more than one sensor can be used in the mobile device, i.e. at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors, GPS, Balistocardiography, and the like. Thus, the mobile device and the device used for carrying out the method of the invention may be physically different devices. In this case, the mobile device may correspond with the device used for carrying out the method of the present invention by any means for data transmission. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Accordingly, for carrying out the method of the present invention, the only requirement is the presence of a dataset of pressure measurements obtained from a subject using a mobile device. The said dataset may also be transmitted or stored from the acquiring mobile device on a permanent or temporary memory device which subsequently can be used to transfer the data to the device used for carrying out the method of the present invention. The remote device which carries out the method of the invention in this setup typically comprises a processor and a database as well as software which is tangibly embedded to said device and, when running on said device, carries out the method of the invention. More typically, the said device may also comprise a user interface, such as a screen, which allows for providing the result of the analysis carried out by the evaluation unit to a user.

The term “predicting” as used herein refers to determining the FVC based on at least one performance parameter determined from measured datasets and a preexisting correlation of this performance parameter and the FVC rather than by measuring the FVC directly. As will be understood by those skilled in the art, such a prediction, although preferred to be, may usually not be correct for 100% of the investigated subjects. The term, however, requires that the FVC can be correctly predicted in a statistically significant portion of subjects. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05. The term also encompasses any kind of diagnosing, monitoring or staging of SMA based on FVC and, in particular, relates to assessing, diagnosing, monitoring and/or staging of any symptom or progression of any symptom associated with SMA.

The term “spinal muscular atrophy (SMA)” as used herein relates to a neuromuscular disease which is characterized by the loss of motor neuron function, typically, in the spinal chord. As a consequence of the loss of motor neuron function, typically, muscle atrophy occurs resulting in an early dead of the affected subjects. The disease is caused by an inherited genetic defect in the SMN1 gene. The SMN protein encoded by said gene is required for motor neuron survival. The disease is inherited in an autosomal recessive manner.

Symptoms associated with SMA include are flexia, in particular, of the extremities, muscle weakness and poor muscle tone, difficulties in completing developmental phases in childhood, as a consequence of weakness of respiratory muscles, breathing problems occurs as well as secretion accumulation in the lung, as well as difficulties in sucking, swallowing and feeding/eating. Four different types of SMA are known.

The infantile SMA or SMA1 (Werdnig-Hoffmann disease) is a severe form that manifests in the first months of life, usually with a quick and unexpected onset (“floppy baby syndrome”). A rapid motor neuron death causes inefficiency of the major body organs, in particular, of the respiratory system, and pneumonia-induced respiratory failure is the most frequent cause of death. Unless placed on mechanical ventilation, babies diagnosed with SMA1 do not generally live past two years of age, with death occurring as early as within weeks in the most severe cases, sometimes termed SMA0. With proper respiratory support, those with milder SMA1 phenotypes accounting for around 10% of SMA1 cases are known to live into adolescence and adulthood.

The intermediate SMA or SMA2 (Dubowitz disease) affects children who are never able to stand and walk but who are able to maintain a sitting position at least some time in their life. The onset of weakness is usually noticed some time between 6 and 18 months. The progress is known to vary. Some people gradually grow weaker over time while others through careful maintenance avoid any progression. Scoliosis may be present in these children, and correction with a brace may help improve respiration. Muscles are weakened, and the respiratory system is a major concern. Life expectancy is somewhat reduced but most people with SMA2 live well into adulthood.

The juvenile SMA or SMA3 (Kugelberg-Welander disease) manifests, typically, after 12 months of age and describes people with SMA3 who are able to walk without support at some time, although many later lose this ability. Respiratory involvement is less noticeable, and life expectancy is normal or near normal.

The adult SMA or SMA4 manifests, usually, after the third decade of life with gradual weakening of muscles that affects proximal muscles of the extremities frequently requiring the person to use a wheelchair for mobility. Other complications are rare, and life expectancy is unaffected.

Typically, SMA in accordance with the present invention is SMA1 (Werdnig-Hoffmann disease), SMA2 (Dubowitz disease), SMA3 (Kugelberg-Welander diseases) or SMA4

SMA is typically diagnosed by the presence of the hypotonia and the absence of reflexes. Both can be measured by standard techniques by the clinician in a hospital including electromyography. Sometimes, serum creatine kinase may be increased as a biochemical parameter. Moreover, genetic testing is also possible, in particular, as prenatal diagnostics or carrier screening. Moreover, a critical parameter in SMA management is the function of the respiratory system. The function of the respiratory system can be, typically, determined by measuring the forced vital capacity of the subject which will be indicative for the degree of impairment of the respiratory system as a consequence of SMA.

The term “forced vital capacity (FVC)” as used herein refers to is the volume in liters of air that can forcibly be blown out after full inspiration by a subject. It is, typically, determined by spirometry in a hospital or at a doctor's residency using spirometric devices.

The term “subject” as used herein relates to animals and, typically, to mammals. In particular, the subject is a primate and, most typically, a human. The subject in accordance with the present invention shall suffer from or shall be suspected to suffer from SMA, i.e. it may already show some or all of the symptoms associated with the said disease and, in particular, respiratory dysfunctions.

The term “at least one” means that one or more performance parameters may be determined in accordance with the invention, i.e. at least two, at least three, at least four, at least five, at least six, at least seven, at least eight, at least nine or at least ten or even more different performance parameters. Thus, there is no upper limit for the number of different performance parameters which can be determined in accordance with the method of the present invention. Typically, however, there will be between one and ten different performance parameters be used. More typically, the parameter(s) are selected from central motor function capabilities and, even more typically, central motor function capabilities that are selected from the group consisting performance parameters derived from datasets of measurements of voice characteristics and datasets of measurements of fine motoric function.

The term “performance parameter” as used herein refers to a parameter which is indicative for the capability of a subject to carry out a certain activity. More typically, the performance parameter is selected from performance parameters indicative for central motor function capabilities. More typically, said performance parameter is determined from datasets of measurements of voice characteristics and fine motoric function. Particular performance parameters to be used in accordance with the present invention are listed elsewhere herein in more detail.

The term “dataset of measurements” refers to the entirety of data which has been acquired by the mobile device from a subject during measurements or any subset of said data useful for deriving the performance parameter.

The at least one performance parameter can be typically determined from datasets of measurements collected from the subject during carrying out the following activities requiring central motor functions function.

The following tests are typically computer-implemented on a data acquisition device such as a mobile device as specified elsewhere herein.

(1) Tests for Central Motor Functions: Draw a Shape Test and Squeeze a Shape Test

The mobile device may be further adapted for performing or acquiring a data from a further test for distal motor function (so-called “draw a shape test”) configured to measure dexterity and distal weakness of the fingers. The dataset acquired from such test allow identifying the precision of finger movements, pressure profile and speed profile.

The aim of the “Draw a Shape” test is to assess fine finger control and stroke sequencing. The test is considered to cover the following aspects of impaired hand motor function: tremor and spasticity and impaired hand-eye coordination. The patients are instructed to hold the mobile device in the untested hand and draw on a touchscreen of the mobile device 6 pre-written alternating shapes of increasing complexity (linear, rectangular, circular, sinusoidal, and spiral; vide infra) with the second finger of the tested hand “as fast and as accurately as possible” within a maximum time of for instance 30 seconds. To draw a shape successfully the patient's finger has to slide continuously on the touchscreen and connect indicated start and end points passing through all indicated check points and keeping within the boundaries of the writing path as much as possible. The patient has maximum two attempts to successfully complete each of the 6 shapes. Test will be alternatingly performed with right and left hand. User will be instructed on daily alternation. The two linear shapes have each a specific number “a” of checkpoints to connect, i.e. “a−1” segments. The square shape has a specific number “b” of checkpoints to connect, i.e. “b−1” segments. The circular shape has a specific number “c” of checkpoints to connect, i.e. “c−1” segments. The eight-shape has a specific number “d” of checkpoints to connect, i.e. “d−1” segments. The spiral shape has a specific number “e” of checkpoints to connect, “e−1” segments. Completing the 6 shapes then implies to draw successfully a total of “(2a+b+c+d+e−6)” segments.

Typical Draw a Shape test performance parameters of interest:

Based on shape complexity, the linear and square shapes can be associated with a weighting factor (Wf) of 1, circular and sinusoidal shapes a weighting factor of 2, and the spiral shape a weighting factor of 3. A shape which is successfully completed on the second attempt can be associated with a weighting factor of 0.5. These weighting factors are numerical examples which can be changed in the context of the present invention.

1. Shape Completion Performance Scores:
a. Number of successfully completed shapes (0 to 6) (ΣSh) per test
b. Number of shapes successfully completed at first attempt (0 to 6) (ΣSh₁)
c. Number of shapes successfully completed at second attempt (0 to 6) (ΣSh₂)
d. Number of failed/uncompleted shapes on all attempts (0 to 12) (ΣF)
e. Shape completion score reflecting the number of successfully completed shapes adjusted with weighting factors for different complexity levels for respective shapes (0 to 10) (Σ[Sh*Wf])
f. Shape completion score reflecting the number of successfully completed shapes adjusted with weighting factors for different complexity levels for respective shapes and accounting for success at first vs second attempts (0 to 10) (Σ[Sh₁*Wf]+Σ[Sh₂*Wf*0.5])
g. Shape completion scores as defined in #1e, and #1f may account for speed at test completion if being multiplied by 30/t, where t would represent the time in seconds to complete the test.
h. Overall and first attempt completion rate for each 6 individual shapes based on multiple testing within a certain period of time: (ΣSh₁)/(ΣSh₁+ΣSh₂+ΣF) and (ΣSh₁+ΣSh₂)/(ΣSh₁+ΣSh₂+ΣF).

2. Segment Completion and Celerity Performance Scores/Measures:
(analysis based on best of two attempts [highest number of completed segments] for each shape, if applicable)
   a. Number of successfully completed segments (0 to [2a+b+c+d+e−6]) (ΣSe) per test
   b. Mean celerity ([C], segments/second) of successfully completed segments: C=ΣSe/t, where t would represent the time in seconds to complete the test (max 30 seconds)
   c. Segment completion score reflecting the number of successfully completed segments adjusted with weighting factors for different complexity levels for respective shapes (Σ[Se*Wf])
   d. Speed-adjusted and weighted segment completion score (Σ[Se*Wf]*30/t), where t would represent the time in seconds to complete the test.
   e. Shape-specific number of successfully completed segments for linear and square shapes (ΣSe_{LS})
   f Shape-specific number of successfully completed segments for circular and sinusoidal shapes (ΣSe_{CS})
   g. Shape-specific number of successfully completed segments for spiral shape (ΣSe_{S})
   h. Shape-specific mean linear celerity for successfully completed segments performed in linear and square shape testing: C_{L}=ΣSe_{LS}/t, where t would represent the cumulative epoch time in seconds elapsed from starting to finishing points of the corresponding successfully completed segments within these specific shapes.
   i. Shape-specific mean circular celerity for successfully completed segments performed in circular and sinusoidal shape testing: C_{C}=/Se_{CS}/t, where t would represent the cumulative epoch time in seconds elapsed from starting to finishing points of the corresponding successfully completed segments within these specific shapes.
   j. Shape-specific mean spiral celerity for successfully completed segments performed in the spiral shape testing: C_{S}=ΣSe_{S}/t, where t would represent the cumulative epoch time in seconds elapsed from starting to finishing points of the corresponding successfully completed segments within this specific shape.

3. Drawing Precision Performance Scores/Measures:
(analysis based on best of two attempts [highest number of completed segments] for each shape, if applicable)
   a. Deviation (Dev) calculated as the sum of overall area under the curve (AUC) measures of integrated surface deviations between the drawn trajectory and the target drawing path from starting to ending checkpoints that were reached for each specific shapes divided by the total cumulative length of the corresponding target path within these shapes (from starting to ending checkpoints that were reached).
   b. Linear deviation (Dev_{L}) calculated as Dev in #3a but specifically from the linear and square shape testing results.
   c. Circular deviation (Dev_{C}) calculated as Dev in #3a but specifically from the circular and sinusoidal shape testing results.
   d. Spiral deviation (Dev_{S}) calculated as Dev in #3a but specifically from the spiral shape testing results.
   e. Shape-specific deviation (Dev₁₋₆) calculated as Dev in #3a but from each of the 6 distinct shape testing results separately, only applicable for those shapes where at least 3 segments were successfully completed within the best attempt.
   f Continuous variable analysis of any other methods of calculating shape-specific or shape-agnostic overall deviation from the target trajectory.

4.) Pressure Profile Measurement
i) Exerted average pressure
ii) Deviation (Dev) calculated as the standard deviation of pressure

The mobile device may be further adapted for performing or acquiring a data from a further test for distal motor function (so-called “squeeze a shape test”) configured to measure dexterity and distal weakness of the fingers. The dataset acquired from such test allow identifying the precision and speed of finger movements and related pressure profiles. The test may require calibration with respect to the movement precision ability of the subject first.

The aim of the Squeeze a Shape test is to assess fine distal motor manipulation (gripping & grasping) & control by evaluating accuracy of pinch closed finger movement. The test is considered to cover the following aspects of impaired hand motor function: impaired gripping/grasping function, muscle weakness, and impaired hand-eye coordination. The patients are instructed to hold the mobile device in the untested hand and by touching the screen with two fingers from the same hand (thumb+second or thumb+third finger preferred) to squeeze/pinch as many round shapes (i.e. tomatoes) as they can during 30 seconds. Impaired fine motor manipulation will affect the performance. Test will be alternatingly performed with right and left hand. User will be instructed on daily alternation.

Typical Squeeze a Shape test performance parameters of interest:

1. Number of Squeezed Shapes
a. Total number of tomato shapes squeezed in 30 seconds (ΣSh)
b. Total number of tomatoes squeezed at first attempt (ΣSh₁) in 30 seconds (a first attempt is detected as the first double contact on screen following a successful squeezing if not the very first attempt of the test)

2. Pinching Precision Measures:
a. Pinching success rate (P_{SR}) defined as ΣSh divided by the total number of pinching (ΣP) attempts (measured as the total number of separately detected double finger contacts on screen) within the total duration of the test.
b. Double touching asynchrony (DTA) measured as the lag time between first and second fingers touch the screen for all double contacts detected.
c. Pinching target precision (P_{TP}) measured as the distance from equidistant point between the starting touch points of the two fingers at double contact to the centre of the tomato shape, for all double contacts detected.
d. Pinching finger movement asymmetry (P_{FMA}) measured as the ratio between respective distances slid by the two fingers (shortest/longest) from the double contact starting points until reaching pinch gap, for all double contacts successfully pinching.
e. Pinching finger velocity (P_{FV}) measured as the speed (mm/sec) of each one and/or both fingers sliding on the screen from time of double contact until reaching pinch gap, for all double contacts successfully pinching.
f Pinching finger asynchrony (P_{FA}) measured as the ratio between velocities of respective individual fingers sliding on the screen (slowest/fastest) from the time of double contact until reaching pinch gap, for all double contacts successfully pinching.
g. Continuous variable analysis of 2a to 2f over time as well as their analysis by epochs of variable duration (5-15 seconds)
h. Continuous variable analysis of integrated measures of deviation from target drawn trajectory for all tested shapes (in particular the spiral and square)

3.) Pressure Profile Measurement
i) Exerted average pressure
ii) Deviation (Dev) calculated as the standard deviation of pressure

More typically, the Squeeze a Shape test and the Draw a Shape test are performed in accordance with the method of the present invention. Even more specifically, the performance parameters listed in the Table 1 below are determined.

The data acquisition device may be further adapted for performing or acquiring a data from a further test for central motor function (so-called “voice test”) configured to measure proximal central motoric functions by measuring voicing capabilities.

Cheer-the-Monster Test:

The term “Cheer-the-Monster test”, as used herein, relates to a test for sustained phonation, which is, in an embodiment, a surrogate test for respiratory function assessments to address abdominal and thoracic impairments, in an embodiment including voice pitch variation as an indicator of muscular fatigue, central hypotonia and/or ventilation problems. In an embodiment, Cheer-the-Monster measures the participant's ability to sustain a controlled vocalization of an “aaah” sound. The test uses an appropriate sensor to capture the participant's phonation, in an embodiment a voice recorder, such as a microphone.

In an embodiment, the task to be performed by the subject is as follows: Cheer the Monster requires the participant to control the speed at which the monster runs towards his goal. The monster is trying to run as far as possible in 30 seconds. Subjects are asked to make as loud an “aaah” sound as they can, for as long as possible. The volume of the sound is determined and used to modulate the character's running speed. The game duration is 30 seconds so multiple “aaah” sounds may be used to complete the game if necessary.

Tap-the-Monster Test:

The term “Tap the Monster test”, as used herein, relates to a test designed for the assessment of distal motor function in accordance with MFM D3 (Bérard C et al. (2005), Neuromuscular Disorders 15:463). In an embodiment, the tests are specifically anchored to MFM tests 17 (pick up ten coins), 18 (go around the edge of a CD with a finger), 19 (pick up a pencil and draw loops) and 22 (place finger on the drawings), which evaluate dexterity, distal weakness/strength, and power. The game measures the participant's dexterity and movement speed.

In an embodiment, the task to be performed by the subject is as follows: Subject taps on monsters appearing randomly at 7 different screen positions.

More typically, the voice test is performed in accordance with the method of the present invention.

In an embodiment, at least one performance parameter selected from the performance parameters listed in Table 1 is determined. In a further embodiment, at least two, at least three, at least four, at least five, at least six, at least seven, at least eight, or at least nine, performance parameters of Table 1 are determined. In a further embodiment, at least three, in a further embodiment at least five, in a further embodiment at least eight, performance parameters of Table 1 are determined. In a further embodiment all performance parameters listed Table 1 are determined.

TABLE 1
Typical performance parameters for central motor function capabilities

| Performance parameter | test | description | rank |
|---|---|---|---|
| lmax_pressure_min | Distal Motor | The minimum value of each | 1 |
| | Function test | maximum pressure reading | |
| | (Tap-The- | per finger tap | |
| | Monster) | | |
| log10 DTA_F | Squeeze-A- | the mean lag time between | 2 |
| | Shape | first and second fingers | |
| | | touch the screen of failed | |
| | | pinches | |
| log10 | Voice test | Mean absolute difference | 3 |
| norm_pct_diff_Mean_MFCCs_9 | | of successive cycles of the | |
| | | 9^{th} Mel Frequency Cepstral | |
| | | Coefficient (MFCC) | |
| log10 std_Mean_MFCCs_8 | Voice test | The standard deviation of | 4 |
| | | the mean value of | |
| | | successive cycles of the 8th | |
| | | MFCC | |
| logistic fatigue_index | Voice test | An estimate for vocal | 5 |
| | | fatigue defined as the ratio | |
| | | of max duration of the first | |
| | | half to max duration of the | |
| | | second half | |
| log10 DTA_S | Squeeze-A- | the mean lag time between | 6 |
| | Shape | first and second fingers | |
| | | touch the screen of | |
| | | successful pinches | |
| sigmoid | Draw-A- | square root of the drawing | 7 |
| LINE_TOP_TO_BOTTOM_errSQRT | Shape | error for the line top-to- | |
| | | bottom shape | |
| log10 DTA_0_15 | Squeeze-A- | the mean lag time between | 8 |
| | Shape | first and second fingers | |
| | | touch the screen between | |
| | | time window 0 s-15 s | |
| log10 DTA_15_30 | Squeeze-A- | the mean lag time between | 9 |
| | Shape | first and second fingers touch | |
| | | the screen between | |
| | | time window 15 s-30 s | |
| log10 DTA | Squeeze-A- | DTA = mean(pinch_start − | 10 |
| | Shape | finger_down): the mean lag | |
| | | time between first and | |
| | | second fingers touch the | |
| | | screen | |

However, in accordance with the method of the present invention, further clinical, biochemical or genetic parameters may be considered. Typically, said further parameters may be obtained from electromyography, measurement of creatine kinase and/or genetic testing for, e.g., SMN1, SMN2 and/or VABP gene mutations and/or aberrations.

The term “mobile device” as used herein refers to any portable device which comprises at least a sensor and data-recording equipment suitable for obtaining the dataset of the aforementiond measurements. This may also require a data processor and storage unit as well as a display for electronically simulating a measurement test on the mobile device. The data processor may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. Moreover, from the activity of the subject data shall be recorded and compiled to a dataset which is to be evaluated by the method of the present invention either on the mobile device itself or on a second device. Depending on the specific setup envisaged, it may be necessary that the mobile device comprises data transmission equipment in order to transfer the acquired dataset from the mobile device to further device. Particular well suited as mobile devices according to the present invention are smartphones, portable multimedia devices or tablet computers. Alternatively, portable sensors with data recording and processing equipment may be used. Further, depending on the kind of activity test to be performed, the mobile device shall be adapted to display instructions for the subject regarding the activity to be carried out for the test. Particular envisaged activities to be carried out by the subject are described elsewhere herein and encompass the tests for central motor function capabilities as described in this specification.

Determining at least one performance parameter can be achieved either by deriving a desired measured value from the dataset as the performance parameter directly. Alternatively, the performance parameter may integrate one or more measured values from the dataset and, thus, may be a derived from the dataset by mathematical operations such as calculations. Typically, the performance parameter is derived from the dataset by an automated algorithm, e.g., by a computer program which automatically derives the performance parameter from the dataset of activity measurements when tangibly embedded on a data processing device feed by the said dataset.

The term “reference” as used herein refers to an identifier, which allows establishing a correlation between the determined at least on performance characteristic and the FVC. The reference is, typically, obtained from a computer-implemented regression model generated on training data, in an embodiment using partial least-squares (PLS) analysis, with the at least one performance parameters. The said training data are, typically, datasets of measurements of central motor function capabilities from subjects suffering from SMA with known FVC. The reference may be a model equation which allows to calculate the FVC to be predicted form the determined at least one performance parameter. Alternatively, it may be a correlation curve or other graphical representation such as a scoring chart, at least one predictions plot, at least one correlations plot, and at least one residuals plot from which the FVC to be predicted can be derived. A regression model may be established by analyzing the training data as referred above by PLS using a processing unit in a data processing device such as a mobile device. The reference is, thus, typically a model equation, a scoring chart, at least one predictions plot, at least one correlations plot, and at least one residuals plot from the analysis, in an embodiment the PLS analysis.

Comparing the determined at least one performance parameter to a reference can be achieved by an automated comparison algorithm implemented on a data processing device such as a computer. The algorithm aims at deriving the predicted FVC from the regression model. This can be done, e.g., by feeding the at least one performance parameter into a model equation or by comparing it to a correlation curve or other graphical representation. As a result of the comparison, the FVC can in the subject can be predicted.

The predicted FVC is subsequently indicated to the subject or another person, such as a medical practitioner. Typically, this is achieved by displaying the predicted FVC on a display of the mobile device or the evaluation device. Alternatively, a recommendation for a therapy, such as a drug treatment, or for a certain life style, e.g. respiration measures, is provided automatically to the subject or other person. To this end, the predicted FVC is compared to recommendations allocated to different FVCs in a database. Once the predicted FVC matches one of the stored and allocated FVCs, a suitable recommendation can be identified due to the allocation of the recommendation to the stored diagnosis matching the predicted FVC. Accordingly, it is, typically, envisaged that the recommendations and FVCs are present in form of a relational database. However, other arrangements which allow for the identification of suitable recommendations are also possible and known to the skilled artisan.

Typically, the method of the present invention for predicting FVC in a subject may be carried out as follows:

First, at least one performance parameter is determined from an existing dataset of measurements of central motor function capabilities obtained from said subject using a mobile device. Said dataset may have been transmitted from the mobile device to an evaluating device, such as a computer, or may be processed in the mobile device in order to derive the at least one performance parameter from the dataset.

Second, the determined at least one performance parameter is compared to a reference by, e.g., using a computer-implemented comparison algorithm carried out by the data processor of the mobile device or by the evaluating device, e.g., the computer. The said reference is obtained from a computer-implemented regression model generated on training data, in an embodiment using partial least-squares (PLS) analysis, with the at least one performance parameters. The result of the comparison is assessed with respect to the reference used in the comparison and based on the said assessment the FVC of the subject will be automatically predicted.

Third, the FVC is indicated to the subject or other person, such as a medical practitioner.

The invention, in light of the above, also specifically contemplates a method of predicting the FVC in a subject suffering from SMA comprising the steps of:
a) obtaining from said subject using a mobile device a dataset of measurements of central motor function capabilities during predetermined activity performed by the subject;
b) determining at least one performance parameter determined from a dataset of measurements obtained from said subject using a mobile device;
c) comparing the determined at least one performance parameter to a reference obtained from a computer-implemented regression model generated on training data, in an embodiment using partial least-squares (PLS) analysis, with the at least one performance parameters; and
d) predicting FVC in said subject.

Advantageously, it has been found in the studies underlying the present invention that performance parameters obtained from datasets of measurements of central motor function capabilities and, in particular, voice characteristics and fine motoric function, in SMA patients can be used as digital biomarkers for predicting the FVC in those patients. The performance parameters can be compared to references obtained from a computer-implemented regression model generated e.g. on training data using partial least-squares (PLS) analysis with the at least one performance parameters. The said datasets can be acquired from the SMA patients in a convenient manner by using mobile devices such as the omnipresent smart phones, portable multimedia devices or tablet computers on which the subjects perform certain tests. The datasets acquired can be subsequently evaluated by the method of the invention for the performance parameter(s) suitable as digital biomarker. Said evaluation can be carried out on the same mobile device or it can be carried out on a separate remote device. Moreover, by using such mobile devices, recommendations on life style or therapy based on the predicted FVC can be provided to the patients directly, i.e. without the consultation of a medical practitioner in a doctor's office or hospital ambulance. Thanks to the present invention, the life conditions of SMA patients can be adjusted more precisely to the actual FVC, i.e. the respiratory status, due to the use of actual determined performance parameters by the method of the invention.

Thereby, therapeutic measures such as drug treatments or respiration support can be selected that are more efficient for the current status of the patient.

The method of the present invention may be used for:
assessing the disease condition;
monitoring patients, in particular, in a real life, daily situation and on large scale;
supporting patients with life style, support and/or therapy recommendations;
investigating drug efficacy, e.g. also during clinical trials;
facilitating and/or aiding therapeutic decision making;
supporting hospital managements;
supporting rehabilitation measure management;
improving the disease condition as a rehabilitation instrument stimulating higher density cognitive, motoric and walking activity
supporting health insurances assessments and management; and/or
supporting decisions in public health management.

The explanations and definitions for the terms made above apply mutatis mutandis to the embodiments described herein below.

In the following, particular embodiments of the method of the present invention are described:

In an embodiment of the method of the invention, said SMA is SMA1 (Werdnig-Hoffmann disease), SMA2 (Dubowitz disease), SMA3 (Kugelberg-Welander diseases) or SMA4.

In yet another embodiment, the said measurements of central motor function capabilities have been carried out using a mobile device.

In an embodiment, said mobile device is comprised in a smartphone, smartwatch, wearable sensor, portable multimedia device or tablet computer.

In yet another embodiment, said measurements of central motor function capabilities comprise measurements of voice characteristics and fine motoric function.

In a further embodiment, at least ten performance parameters are used.

In an another embodiment, said reference obtained from a computer-implemented regression model generated on training data, in an embodiment using partial least-squares (PLS) analysis, with the at least one performance parameters is a model equation, a scoring chart, at least one predictions plot, at least one correlations plot, and at least one residuals plot from the analysis, in an embodiment the PLS analysis.

The present invention also contemplates a computer program, computer program product or computer readable storage medium having tangibly embedded said computer program, wherein the computer program comprises instructions when run on a data processing device or computer carry out the method of the present invention as specified above. Specifically, the present disclosure further encompasses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a dataset of pressure measurements obtained from the subject using a mobile, and
a data stream signal, typically encrypted, comprising the at least one performance parameter derived from the dataset of pressure measurements obtained from the subject using a mobile.

The present invention, further, relates to a method for determining at least one performance parameter from a dataset of measurements of central motor function capabilities from said subject suffering from SMA using a mobile device
a) deriving at least one performance parameter from a dataset of measurements of central motor function capabilities from said subject using a mobile device; and
b) comparing the determined at least one performance parameter to a reference, said reference being obtained from a computer-implemented regression model generated on training data, in an embodiment using partial least-squares (PLS) analysis, with the at least one performance parameters,
   wherein, typically, said at least one performance parameter can aid predicting the FVC in said subject.

The present invention also encompasses a method for determining efficacy of a therapy against SMA comprising the steps of the method of the invention (i.e. the method for predicting FVC) and the further step of determining a therapy response if improvement of SMA and/or FVC occurs in the subject upon therapy or determining a failure of response if worsening of SMA and/or FVC occurs in the subject upon therapy or if SMA and/or FVC remains unchanged.

The term “a therapy against a SMA” as used herein refers to all kinds of medical treatments, including drug-based therapies, respiratory support and the like. The term also encompasses, life-style recommendations and rehabilitation measures. Typically, the method encompasses recommendation of a drug-based therapy and, in particular, a therapy with a drug known to be useful for the treatment of SMA. Such drug may be Nusinersen, butyrates, valproic acid, hydroxyurea or riluzole. Moreover, the aforementioned method may comprise in yet an embodiment the additional step of applying the recommended therapy to the subject.

Moreover, encompassed in accordance with the present invention is a method for determining efficacy of a therapy against SMA comprising the steps of the aforementioned method of the invention (i.e. the method for predicting FVC) and the further step of determining a therapy response if improvement of SMA and/or FVC occurs in the subject upon therapy or determining a failure of response if worsening of SMA and/or FVC occurs in the subject upon therapy or if SMA and/or FVC remains unchanged.

The term “improvement” as referred to in accordance with the present invention relates to any improvement of the overall disease condition or of individual symptoms thereof and, in particular, the predicted FVC. Likewise, a “worsening” means any worsening of the overall disease condition or individual symptoms thereof and, in particular, the predicted FVC. Since, SMA as a progressing disease is associated typically with a worsening of the overall disease condition and symptoms thereof, the worsening referred to in connection with the aforementioned method is an unexpected or untypical worsening which goes beyond the normal course of the disease. Unchanged SMA means that the overall disease condition and the symptoms accompanying it are within the normal course of the disease.

Moreover, the present invention pertains to a method of monitoring SMA in a subject comprising determining whether said disease improves, worsens or remains unchanged in a subject by carrying out the steps of the method of the invention (i.e. the method of predicting FVC) at least two times during a predefined monitoring period. If the FVC improves, the disease improves, if the FVC worsens, the disease worsens and if the FVC remains unchanged, the disease does as well.

The present invention relates to a mobile device comprising a processor, at least one sensor and a database as well as software which is tangibly embedded to said device and, when running on said device, carries out the method of the present invention.

The said mobile device is, thus, configured to be capable of acquiring the dataset and to determine the performance parameter therefrom. Moreover, it is configured to carry out the comparison to a reference and to establish the prediction, i.e. the prediction of the FVC. Moreover, the mobile device may, typically, also be capable of obtaining and/or generating the reference from a computer-implemented regression model generated on training data, in an embodiment using partial least-squares (PLS) analysis, with the at least one performance parameters. Further details on how the mobile device can be designed for said purpose have been described elsewhere herein already in detail.

A system comprising a mobile device comprising at least one sensor and a remote device comprising a processor and a database as well as software which is tangibly embedded to said device and, when running on said device, carries out the method of the invention, wherein said mobile device and said remote device are operatively linked to each other.

Under “operatively linked to each other” it is to be understood that the devices are connect as to allow data transfer from one device to the other device. Typically, it is envisaged that at least the mobile device which acquires data from the subject is connect to the remote device carrying out the steps of the methods of the invention such that the acquired data can be transmitted for processing to the remote device. However, the remote device may also transmit data to the mobile device such as signals controlling or supervising its proper function. The connection between the mobile device and the remote device may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced, 5G, or Bluetooth. Further details may be found elsewhere in this specification. For data acquisition, the mobile device may comprise a user interface such as screen or other equipment for data acquisition. Typically, the activity measurements can be performed on a screen comprised by a mobile device, wherein it will be understood that the said screen may have different sizes including, e.g., a 5.1 inch screen.

Moreover, it will be understood that the present invention contemplates the use of the mobile device or the system according to the present invention for predicting the forced vital capacity (FVC) in a subject suffering from spinal muscular atrophy (SMA) using at least one performance parameter from a dataset of measurements of central motor function capabilities from said subject.

The present invention also contemplates the use of the mobile device or the system according to the present invention for monitoring patients, in particular, in a real life, daily situation and on large scale.

Encompassed by the present invention is furthermore the use of the mobile device or the system according to the present invention for supporting patients with life style and/or therapy recommendations.

Yet, it will be understood that the present invention contemplates the use of the mobile device or the system according to the present invention for investigating drug safety and efficacy, e.g. also during clinical trials.

Further, the present invention contemplates the use of the mobile device or the system according to the present invention for facilitating and/or aiding therapeutic decision making.

Furthermore, the present invention provides for the use of the mobile device or the system according to the present invention for improving the disease condition as a rehabilitation instrument, and for supporting hospital management, rehabilitation measure management, health insurances assessments and management and/or supporting decisions in public health management.

In the following, further particular embodiments of the invention are listed:

Embodiment 1: A method for predicting the forced vital capacity (FVC) in a subject suffering from spinal muscular atrophy (SMA) comprising the steps of:
a) determining at least one performance parameter from a dataset of measurements of central motor function capabilities from said subject;
b) comparing the determined at least one performance parameter to a reference obtained from a computer-implemented regression model generated on training data, in an embodiment using partial least-squares (PLS) analysis, with the at least one performance parameters; and
c) predicting the FVC of the subject based on said comparison.

Embodiment 2: The method of embodiment 1, wherein said SMA is SMA1 (Werdnig-Hoffmann disease), SMA2 (Dubowitz disease), SMA3 (Kugelberg-Welander diseases) or SMA4.

Embodiment 3: The method of embodiment 1 or 2, wherein the said measurements of central motor function capabilities have been carried out using a mobile device, in an embodiment wherein the said measurements of central motor function capabilities are carried out using a mobile device.

Embodiment 4: The method of embodiment 3, wherein said mobile device is comprised in a smartphone, smartwatch, wearable sensor, portable multimedia device or tablet computer.

Embodiment 5: The method of any one of embodiments 1 to 4, wherein said measurements of central motor function capabilities comprise measurements of voice characteristics and fine motoric function.

Embodiment 6: The method of any one of embodiments 1 to 5, wherein at least ten performance parameters are used, in an embodiment the ten performance parameters listed in Table 1.

Embodiment 7: The method of any one of embodiments 1 to 6, wherein at least three, in an embodiment at least four, in a further embodiment at least six, performance parameters of Table 1 are used, in an embodiment wherein at least the first three, in an embodiment the first four, in a further embodiment the first six, performance parameters of Table 1 are used.

Embodiment 8: The method of any one of embodiments 1 to 7, wherein all performance parameters of Table 1 are used.

Embodiment 9: The method of any one of embodiments 1 to 8, wherein the at least one performance parameter of step a) is derived from the dataset by an automated algorithm tangibly embedded on a data processing device.

Embodiment 10: The method of any one of embodiments 1 to 11, wherein comparing the at least one performance parameter to a reference in step b) is achieved by an automated comparison algorithm implemented on a data processing device.

Embodiment 11: The method of any one of embodiments 1 to 6, wherein said reference obtained from a computer-implemented regression model generated on training data, in an embodiment using partial least-squares (PLS) analysis, with the at least one performance parameters is a model equation, a scoring chart, at least one predictions plot, at least one correlations plot, and at least one residuals plot from the analysis, in an embodiment the PLS analysis.

Embodiment 12: The method of any one of embodiments 1 to 11, wherein said method is computer-implemented.

Embodiment 13: The method of any one of embodiments 1 to 12, wherein said performance parameter is indicative for the capability of a subject to carry out a certain activity, in an embodiment is selected from performance parameters indicative for central motor function capabilities, in a further embodiment is determined from datasets of measurements of voice characteristics and fine motoric function, in a further embodiment is a performance parameter of Table 1.

Embodiment 14: A mobile device comprising a processor, at least one sensor and a database as well as software which is tangibly embedded to said device and, when running on said device, carries out at least step a) of the method of any one of embodiments 1 to 13, in an embodiment carries out the method of any one of embodiments 1 to 13.

Embodiment 15: A system comprising a mobile device comprising at least one sensor and a remote device comprising a processor and a database as well as software which is tangibly embedded to said device and, when running on said device, carries out the method of any one of embodiments 1 to 13, wherein said mobile device and said remote device are operatively linked to each other.

Embodiment 16: Use of the mobile device according to embodiment 14 or the system of embodiment 15 for predicting the forced vital capacity (FVC) in a subject suffering from spinal muscular atrophy (SMA) using at least one performance parameter from a dataset of measurements of central motor function capabilities from said subject.

All references cited throughout this specification are herewith incorporated by reference with respect to their entire disclosure content and with respect to the specific disclosure contents mentioned in the specification.

### BRIEF DESCRIPTION OF THE FIGURES

FIG. 1 shows FVC prediction results obtained with different models, i.e. k nearest neighbors (kNN); linear regression; partial last-squares (PLS); random forest (RF); and extremely randomized Trees (XT); f: number of features included in model, y-axis: rₛ (correlation between predicted and actual values); upper row: test data set, lower row: training data; in the lower row, upper graphs relate to “mean” prediction, i.e. the prediction on the average value of all observations per subject, and the lower graphs relate to “all” prediction, i.e. prediction on all individual observations; the best result is obtained using PLS.

### EXAMPLES

The following Examples merely illustrate the invention. Whatsoever, they shall not be construed in a way as to limit the scope of the invention.

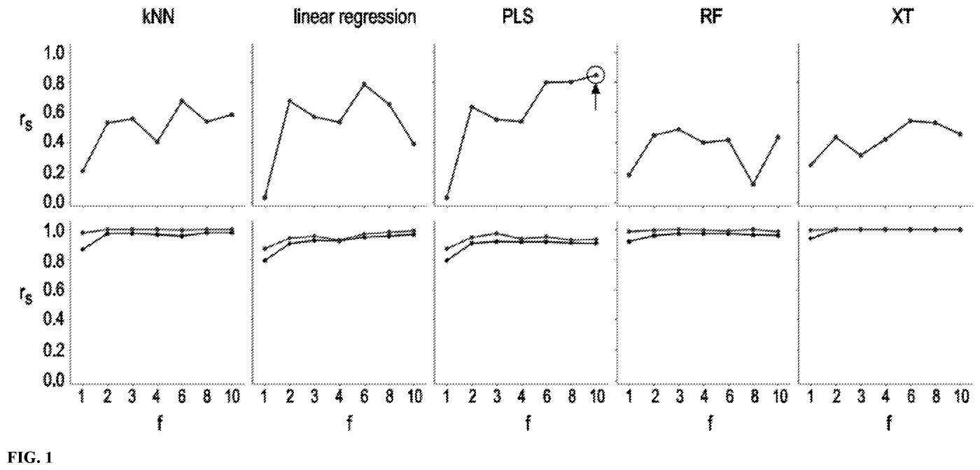

Example 1: Data from a study (“OLEOS”) including 14 subjects were investigated by kNN, linear regression, PLS, RF and XT. In total, 1326 features from 9 tests were evaluated during model building. Relevant tests and and parameters determined are described below in Table 2. The models built by the different techniques were investigated by a machine learning algorithm in order to identify the model with the best correlation. FIG. 1 shows a correlations plot for analysis models, in particular regression models, for predicting an FVC value indicative of SMA. FIG. 1, in particular, shows the Spearman correlation coefficient rs between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. It was found that the best performing regression model is PLS with 10 features included in the model, having an rs value of 0.81, indicated with circle and arrow. The following table (Table 2) gives an overview for features from the PLS algorithm (best correlation) test from which the feature was derived, short description of feature and ranking:

TABLE 2

| Performance parameter | test | description | rank |
|---|---|---|---|
| lmax_pressure_min | Distal Motor | The minimum value of each | 1 |
| | Function test | maximum pressure reading | |
| | (Tap-The- | per finger tap | |
| | Monster) | | |
| log10 DTA_F | Squeeze-A- | the mean lag time between | 2 |
| | Shape | first and second fingers | |
| | | touch the screen of failed | |
| | | pinches | |
| log10 | Voice test | Mean absolute difference | 3 |
| norm_pct_diff_Mean_MFCCs_9 | | of successive cycles of the | |
| | | 9^{th} Mel Frequency Cepstral | |
| | | Coefficient (MFCC) | |
| log10 std_Mean_MFCCs_8 | Voice test | The standard deviation of | 4 |
| | | the mean value of | |
| | | successive cycles of the 8th | |
| | | MFCC | |
| logistic fatigue_index | Voice test | An estimate for vocal | 5 |
| | | fatigue defined as the ratio | |
| | | of max duration of the first | |
| | | half to max duration of the | |
| | | second half | |
| log10 DTA_S | Squeeze-A- | the mean lag time between | 6 |
| | Shape | first and second fingers | |
| | | touch the screen of | |
| | | successful pinches | |
| sigmoid | Draw-A- | square root of the drawing | 7 |
| LINE_TOP_TO_BOTTOM_errSQRT | Shape | error for the line top-to- | |
| | | bottom shape | |
| log10 DTA_0_15 | Squeeze-A- | the mean lag time between | 8 |
| | Shape | first and second fingers | |
| | | touch the screen between | |
| | | time window 0 s-15 s | |
| log10 DTA_15_30 | Squeeze-A- | the mean lag time between | 9 |
| | Shape | first and second fingers | |
| | | touch the screen between | |
| | | time window 15 s-30 s | |
| log10 DTA | Squeeze-A- | DTA = mean(pinch_start − | 10 |
| | Shape | finger_down): the mean lag | |
| | | time between first and | |
| | | second fingers touch the | |
| | | screen | |

### CITED REFERENCES

Bérard C et al. (2005), Neuromuscular Disorders 15:463.

Brzustowicz 1990, Nature. 344 (6266): 540-541.

Lefebvre 1995, Cell. 80 (1): 155-165.

Rutkove 2010, Muscle & Nerve. 42 (6): 915-921.

1. A method for predicting the forced vital capacity (FVC) in a subject suffering from spinal muscular atrophy (SMA) comprising the steps of:
a) determining at least one performance parameter from a dataset of measurements of central motor function capabilities from said subject;
b) comparing the determined at least one performance parameter to a reference obtained from a computer-implemented regression model generated on training data using partial least-squares (PLS) analysis with the at least one performance parameters; and
c) predicting the FVC of the subject based on said comparison.

2. The method of claim 1, wherein said SMA is SMA1 (Werdnig-Hoffmann disease), SMA2 (Dubowitz disease), SMA3 (Kugelberg-Welander diseases) or SMA4.

3. The method of claim 1, wherein the said measurements of central motor function capabilities have been carried out using a mobile device.

4. The method of claim 3, wherein said mobile device is comprised in a smartphone, smartwatch, wearable sensor, portable multimedia device or tablet computer.

5. The method of claim 1, wherein said measurements of central motor function capabilities comprise measurements of voice characteristics and fine motoric function.

6. The method of claim 1, wherein at least ten performance parameters are used.

7. The method of claim 1, wherein at least three performance parameters of Table 1 are used.

8. The method of claim 1, wherein all performance parameters of Table 1 are used.

9. The method claim 1, wherein the at least one performance parameter of step a) is derived from the dataset by an automated algorithm tangibly embedded on a data processing device.

10. The method of claim 1, wherein comparing the at least one performance parameter to a reference in step b) is achieved by an automated comparison algorithm implemented on a data processing device.

11. The method of claim 1, wherein said reference obtained from a computer-implemented regression model generated on training data using partial least-squares (PLS) analysis with the at least one performance parameters is a model equation, a scoring chart, at least one predictions plot, at least one correlations plot, and at least one residuals plot from the PLS analysis.

12. The method of claim 1, wherein said method is computer-implemented.

13. The method of claim 1, wherein said performance parameter is indicative for the capability of a subject to carry out a certain activity, in an embodiment is selected from performance parameters indicative for central motor function capabilities, in a further embodiment is determined from datasets of measurements of voice characteristics and fine motoric function, in a further embodiment is a performance parameter of Table 1.

14. A mobile device comprising a processor, at least one sensor and a database as well as software which is tangibly embedded to said device and, when running on said device, carries out at least step a) of the method of claim 1.

15. A system comprising a mobile device comprising at least one sensor and a remote device comprising a processor and a database as well as software which is tangibly embedded to said device and, when running on said device, carries out the method of claim 1, wherein said mobile device and said remote device are operatively linked to each other.

16. Use of the mobile device according to claim 14 for predicting the forced vital capacity (FVC) in a subject suffering from spinal muscular atrophy (SMA) using at least one performance parameter from a dataset of measurements of central motor function capabilities from said subject.
